(19) United States
(12) Patent Application Publication
(10) Pub. No.: US 2018/0028148 A1
(43) Pub. Date: Feb. 1, 2018

(54) ULTRASOUND DIAGNOSIS APPARATUS, MEDICAL IMAGE PROCESSING APPARATUS, AND MEDICAL IMAGE PROCESSING METHOD

(71) Applicant: Toshiba Medical Systems Corporation, Otawara-shi (JP)

(72) Inventors: Koji Ando, Otawara (JP); Nobuhide Ooi, Nasushiobara (JP); Mitsuo Akiyama, Otawara (JP)

(73) Assignee: Toshiba Medical Systems Corporation, Otawara-shi (JP)

(21) Appl. No.: 15/658,988

(22) Filed: Jul. 25, 2017

(30) Foreign Application Priority Data
Jul. 26, 2016 (JP) 2016-146441
Jul. 21, 2017 (JP) 2017-141785

(51) Int. Cl. A61B 8/14; A61B 8/08; A61B 8/00

(52) U.S. Cl. CPC A61B 8/145; A61B 8/463; A61B 8/5207; A61B 8/565

(57) ABSTRACT

According to one embodiment, an ultrasound diagnosis apparatus includes an acquisition unit and an individual image generating unit. The acquisition unit acquires multiple cross-sectional image data that are ultrasound image data for simultaneously displaying a plurality of cross sections of a subject. The individual image generating unit generates individual image data for each of the cross sections based on the position information of the cross section along with the display of the multiple cross-sectional image data.

### CROSS-REFERENCE TO RELATED APPLICATION(S)

This application is based upon and claims the benefit of priority from Japanese Patent Application No. 2016-146441, filed Jul. 26, 2016; No. 2017-141785, filed Jul. 21, 2017; the entire contents of (all of) which are incorporated herein by reference.

### FIELD

Embodiments described herein relate generally to an ultrasound diagnosis apparatus, a medical image processing apparatus, and a medical image processing method.

### BACKGROUND

A multi-plane probe is sometimes used in an examination using an ultrasound diagnosis apparatus. The multi-plane probe is an ultrasound probe capable of concurrently acquiring image data of a plurality of cross sections simultaneously by an operator such as a doctor or a sonographer.

Examples of the examination to which application of the multi-plane probe is desired include a stress echo examination. In the stress echo examination, images before and after a stress is applied to a subject are acquired to be compared and analyzed. Examples of the stress include exercises such as step exercises and drug administration.

Examples of the site images of which are acquired include the heart. By comparatively analyzing the images of the heart before and after the stress application, the cardiac function of the subject is evaluated. For acquiring the images of the heart before and after the heart rate changes due to the stress application, it is desired to acquire image data of a plurality of cross sections simultaneously in a short time by using the multi-plane probe. Normally, the image data of the cross sections are sequentially displayed on a monitor along with the acquisition of the image data. This enables the operator to operate the ultrasound diagnosis apparatus and the ultrasound probe while checking whether image data of desired cross sections are acquired.

At this time, on the display, the image data of the cross sections are displayed side by side in one display window. For example, when image data of two cross sections are acquired (biplane), two pieces of cross-sectional image data acquired simultaneously in parallel (in the same time phase) are displayed side by side in one display window. Then, the image data of the cross sections are stored in one set with respect to each time phase in which they are acquired simultaneously.

The image data of the cross sections are usually compared and analyzed by an image processing apparatus such as a workstation. A general workstation, which is not provided with dedicated application software corresponding to the image data acquired by the multi-plane probe, is configured to process image data in the form in which one cross-sectional image data is displayed in one display window.

Therefore, it has been difficult to directly compare and analyze one set of image data (the image data acquired by the multi-plane probe), i.e., the pieces of the cross-sectional image data displayed in one display window. For example, when the operator visually checks a plurality of cross-sectional images included in one set of acquired image data and distinguishes cross-sectional image areas individually by manual operation or the like before the comparative analysis, the work procedure is cumbersome and takes time.

### BRIEF DESCRIPTION OF THE DRAWINGS

FIG. 1 is a block diagram illustrating a configuration of an ultrasound diagnosis apparatus according to a first embodiment;

FIG. 2 is a schematic diagram illustrating the concept of multiple cross-sectional image data and individual image data;

FIG. 3 is a flowchart illustrating the operation of the ultrasound diagnosis apparatus of the first embodiment;

FIG. 4 is a block diagram illustrating a configuration of an ultrasound diagnosis apparatus according to a second embodiment; and

FIG. 5 is a flowchart illustrating the operation of a medical image processing apparatus of the second embodiment.

### DETAILED DESCRIPTION

In general, according to one embodiment, an ultrasound diagnosis apparatus includes an acquisition unit and an individual image generating unit. The acquisition unit acquires multiple cross-sectional image data that are ultrasound image data for simultaneously displaying a plurality of cross sections of a subject. The individual image generating unit generates individual image data for each of the cross sections based on the position information of the cross section along with the display of the multiple cross-sectional image data.

Referring now to the drawings, a description is given of an ultrasound diagnosis apparatus, a medical image processing apparatus, and a medical image processing method according to embodiments.

### First Embodiment

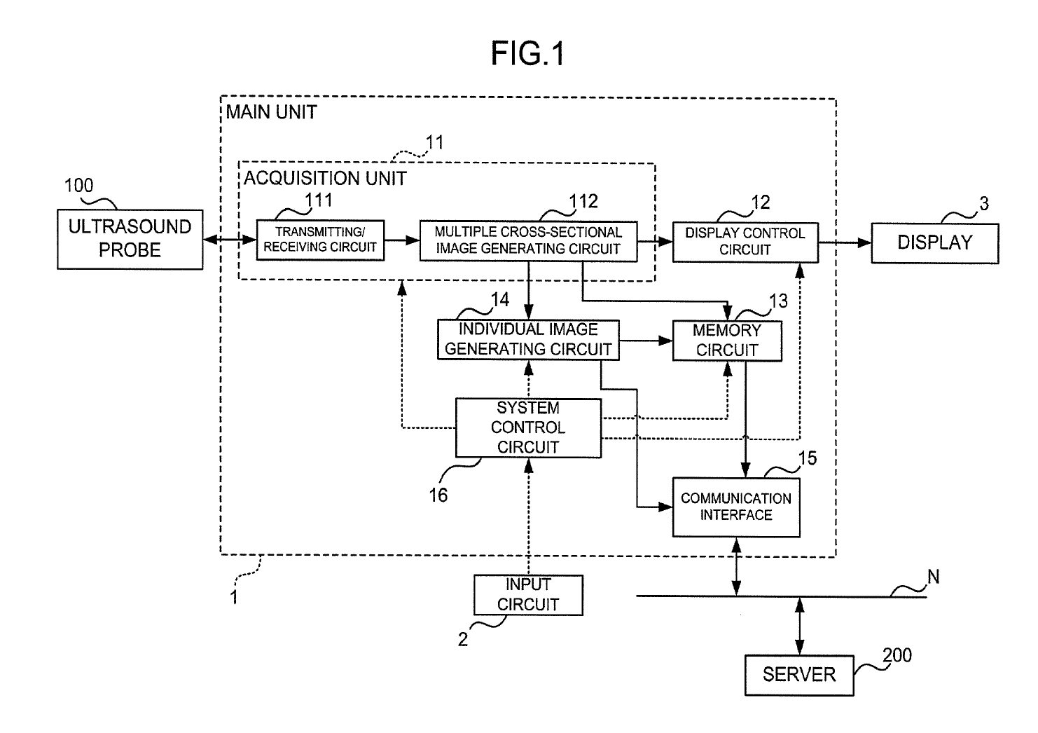

The first embodiment involves an ultrasound diagnosis apparatus. FIG. 1 is a block diagram illustrating a configuration of an ultrasound diagnosis apparatus according to a first embodiment. The ultrasound diagnosis apparatus of the embodiment includes a main unit 1, an input circuit 2, and a display 3, and is communicably connected to an ultrasound probe 100 and a server 200.

A multi-plane probe may be used as the ultrasound probe 100. Examples of the multi-plane probe incudes a two-dimensional array probe having a plurality of ultrasound transducers arrayed two-dimensionally. The ultrasound probe 100 is driven by a transmitting/receiving circuit 111 (described later) to simultaneously scan a plurality of cross sections of a subject. The ultrasound probe 100 outputs an echo signal obtained by the scanning to the transmitting/receiving circuit 111.

The input circuit 2 receives operation by an operator such as a doctor, a sonographer, or the like, and outputs a signal corresponding to the content of the operation to a system control circuit 16. For example, the input circuit 2 includes a trackball, a switch button, a mouse, a keyboard, a touch command screen, a sensitivity time control (STC) slide volume, and the like. The input circuit 2 is an example of the operation unit in the claims.

The display 3 displays a variety of types of ultrasound images under the control of a display control circuit 12. The display 3 is communicably connected to the main unit 1. The display 3 is formed of a display device such as a liquid crystal display (LCD) or an organic electro-luminescence (EL) display.

The main unit 1 includes an acquisition unit 11, the display control circuit 12, a memory circuit 13, an individual image generating circuit 14, a communication interface 15, and the system control circuit 16. The acquisition unit 11 acquires a plurality of pieces of cross-sectional image data (multiple cross-sectional image data), which are ultrasound image data for simultaneously displaying a plurality of cross sections of a subject. The acquisition unit 11 includes the transmitting/receiving circuit 111 and a multiple cross-sectional image generating circuit 112.

The transmitting/receiving circuit 111 is a processor that outputs a pulse signal to the ultrasound probe 100 to thereby generate ultrasound waves. The transmitting/receiving circuit 111 includes a pulser for each channel corresponding to each of the ultrasound transducers, and outputs a pulse signal for each channel so that a plurality of cross sections of a subject are scanned simultaneously. A control program for scanning the cross sections may be set appropriately. In addition, the transmitting/receiving circuit 111 receives an echo signal from the ultrasound probe 100. The transmitting/receiving circuit 111 amplifies the echo signal from the ultrasound probe 100 with respect to each channel based on a set gain, and outputs it to the multiple cross-sectional image generating circuit 112.

The multiple cross-sectional image generating circuit 112 is a processor that generates multiple cross-sectional image data based on the echo signal from the transmitting/receiving circuit 111. Regarding data format, the multiple cross-sectional image data may be ultrasound raster data (hereinafter, RAW data) or pixel value data obtained by converting the RAW data into pixel values. Further, the multiple cross-sectional image data may be two-dimensional image data or three-dimensional image data. A control program for converting the echo signal into the multiple cross-sectional image data may be set appropriately based on a general multi-plane scan program.

The term “processor” as used herein refers to a circuit such as, for example, a central processing unit (CPU), a graphics processing unit (GPU), an application specific integrated circuit (ASIC), a programmable logic device including a simple programmable logic device (SPLD) and a complex programmable logic device (CPLD), a field programmable gate array (FPGA), or the like. The processor reads programs out of the memory circuit and executes them to thereby realize the functions. The programs need not necessarily be stored in the memory circuit, but may be directly incorporated in the circuit of the processor. In this case, the processor realizes the functions by reading and executing the programs incorporated in the circuit. Each processor of the embodiment need not necessarily be configured as a single circuit. A plurality of independent circuits may be combined to form a single processor for implementing the functions. Besides, a plurality of constituent elements in FIG. 1 may be integrated into one processor to realize the functions.

The multiple cross-sectional image generating circuit 112 adds cross-section number information indicating the number of cross sections illustrated and position information indicating the position of each cross section illustrated to the multiple cross-sectional image data. For example, in the case of a biplane image, the cross-section number information indicates “2” as the number of cross sections, and the position information indicates the cross-sectional positions of the two cross sections individually. The position information may be cross-section direction information indicating the direction of each cross section (angle relative to the ultrasound probe 100), or coordinate information indicating each cross section in a three-dimensional coordinate system. In other words, the multiple cross-sectional image generating circuit 112 may generate the multiple cross-sectional image data while adding the cross-section direction information thereto as the position information or may generate the multiple cross-sectional image data while adding the coordinate information thereto as the position information. Besides, the multiple cross-sectional image generating circuit 112 adds the position information to the multiple cross-sectional image data while associating each cross section indicated by the position information with corresponding cross-sectional image data. As a result, each cross-sectional image data in the multiple cross-sectional image data is associated with corresponding position information.

In addition, information such as acquisition date and time, apparatus ID, and the like is also added to the multiple cross-sectional image data. The information added to the multiple cross-sectional image data, such as the position information, the acquisition date and time, the apparatus ID and the like, is herein referred to as supplementary information. The supplementary information may be contained in the header area or may be contained in the source area of the multiple cross-sectional image data.

In this way, one piece of the multiple cross-sectional image data in which a plurality of cross-sectional images are grouped into one set is generated. The multiple cross-sectional image generating circuit 112 outputs the multiple cross-sectional image data to the display control circuit 12. The multiple cross-sectional image generating circuit 112 may output the multiple cross-sectional image data to the memory circuit 13 to store it therein.

The display control circuit 12 is a processor that converts the multiple cross-sectional image data into coordinates for display, and displays the coordinates on the display 3. At this time, similarly to general multi-plane images, the display control circuit 12 displays a plurality of cross-sectional images acquired simultaneously and concurrently in one display window on the display 3. For example, in the case of biplane scan, two cross-sectional images acquired simultaneously and concurrently are displayed side by side in one display window.

The memory circuit 13 has a memory area and is formed of a storage device such as a read only memory (ROM), a random access memory (RAM), and the like. The database structure of the memory area may be set appropriately. The memory circuit 13 is an example of the storage in the claims.

When having received a predetermined setting instruction through the input circuit 2, the multiple cross-sectional image generating circuit 112 outputs the multiple cross-sectional image data to the individual image generating circuit 14. The setting instruction is provided by operation on the input circuit 2, and input operation to a predetermined button switch or the like is set in advance to provide the setting instruction.

Note that either of the multiple cross-sectional image generating circuit 112 or the display control circuit 12 may perform digital scan conversion for converting the coordinate system of the RAW data to the coordinates for display and data conversion for converting the RAW data to the pixel value data.

Each process in the ultrasound probe 100, the transmitting/receiving circuit 111, the multiple cross-sectional image generating circuit 112, the display control circuit 12, and the display 3 is sequentially updated during data acquisition for imaging a plurality of cross sections of a subject simultaneously and concurrently. The update timing is set appropriately as a predetermined frame rate, signal processing rate, or the like. Thereby, for example, during data acquisition in ultrasound stress echocardiography, a plurality of cross sections of a target site are imaged simultaneously and concurrently, and displayed on the display 3. Thus, the operator can view the cross-sectional images displayed on the display 3, and select an image for comparative analysis. A selection signal indicating the selection operation is input to the system control circuit 16 via the input circuit 2. The system control circuit 16 controls the multiple cross-sectional image generating circuit 112 to output multiple cross-sectional image data selected to the individual image generating circuit 14.

The individual image generating circuit 14 is a processor that generates individual image data for each cross section based on position information for each cross section related to the multiple cross-sectional image data. When cross-section direction information is added to the multiple cross-sectional image data as the position information, the individual image generating circuit 14 generates the individual image data with reference to the cross-section direction information. Meanwhile, when coordinate information is added to the multiple cross-sectional image data as the position information, the individual image generating circuit 14 generates the individual image data with reference to the coordinate information.

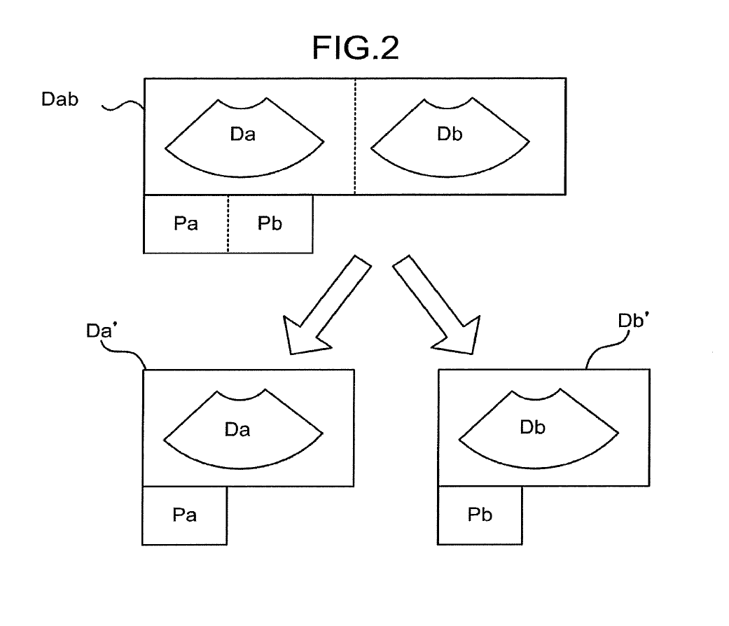

FIG. 2 is a schematic diagram illustrating the concept of the multiple cross-sectional image data and the individual image data. Described below is a case of biplane where the number of cross sections is “2”. The individual image generating circuit 14 receives the multiple cross-sectional image data Dab from the multiple cross-sectional image generating circuit 112. In the multiple cross-sectional image data Dab, image data Da indicating a cross section a, image data Db indicating a cross section b, position information Pa indicating the position of the cross section a, and position information Pb indicating the position of the cross section b are grouped into one set. The image data Da of the cross section a and the image data Db of the cross section b are image data of different cross sections (i.e., the cross section a and the cross section b) simultaneously and concurrently acquired. As described above, the image data Da and the position information Pa are associated with each other in the multiple cross-sectional image data Dab. Similarly, the image data Db and the position information Pb are associated with each other in the multiple cross-sectional image data Dab.

The individual image generating circuit 14 specifies the image data Da associated with the position information Pa from the multiple cross-sectional image data Dab with reference to the position information Pa of the multiple cross-sectional image data Dab. In addition, the individual image generating circuit 14 specifies the image data Db associated with the position information Pb from the multiple cross-sectional image data Dab with reference to the position information Pb of the multiple cross-sectional image data Dab. Even when there are three or more cross sections, the individual image generating circuit 14 can also specify the number of cross sections contained in the multiple cross-sectional image data, the position of each cross section, image data associated with each cross section by referring to the position information.

The individual image generating circuit 14 generates individual image data Da′ with accompanying the position information Pa associated with the image data Da specified. Besides, the individual image generating circuit 14 generates individual image data Db′ with accompanying the position information Pb associated with the image data Db specified. The individual image generating circuit 14 outputs the individual image data Da′ and the individual image data Db′ thus generated to the memory circuit 13 and the communication interface 15. At this time, the individual image generating circuit 14 may output the multiple cross-sectional image data Dab together with the individual image data Da′ and the individual image data Db′ to the memory circuit 13 and the communication interface 15. The individual image data (Da′ and Db′) are generated in conjunction with a simple operation of selecting desired multiple cross-sectional image data Dab. As the multiple cross-sectional image data is converted into the data format of the individual image data (Da′ and Db′), one cross-sectional image is displayed in one display window, which facilitates comparative analysis in a general-purpose work station

An example has been described in which an operator selects desired multiple cross-sectional image data from pieces of multiple cross-sectional image data sequentially generated, and individual image data is generated from the multiple cross-sectional image data selected. Alternatively, each time multiple cross-sectional image data is generated (e.g., for each frame rate), the individual image generating circuit 14 may generate individual image data from the multiple cross-sectional image data. In this case, the individual image generating circuit 14 performs the above-described process each time multiple cross-sectional image data is generated, and sequentially generates individual image data. The individual image generating circuit 14 sequentially outputs the individual image data to the memory circuit 13 and the communication interface 15.

The individual image generating circuit 14 may be configured to generate individual image data when having received a predetermined setting instruction through the input circuit 2. With this, the operator can operate the ultrasound diagnosis apparatus while switching the individual image generating circuit 14 depending on whether it is desired to generate individual image data or it is not necessary to generate individual image data.

The memory circuit 13 stores the individual image data Da′ and the individual image data Db′. The communication interface 15 is communicably connected to the external server 200 via a network N. The communication interface 15 outputs the individual image data Da′ and the individual image data Db′ to the server 200. The communication interface 15 may also output the multiple cross-sectional image data Dab with the data Da′ and Db′ to the server 200. The communication protocols in the communication interface 15, the network N, and the server 200 and the database structure of the server 200 may be appropriately determined. For example, a predetermined Digital Imaging and Communications in Medicine (DICOM) tag is attached to the individual image data Da′ and the individual image data Db′ stored in the server 200 as appropriate. The DICOM tag may contain the above-described position information.

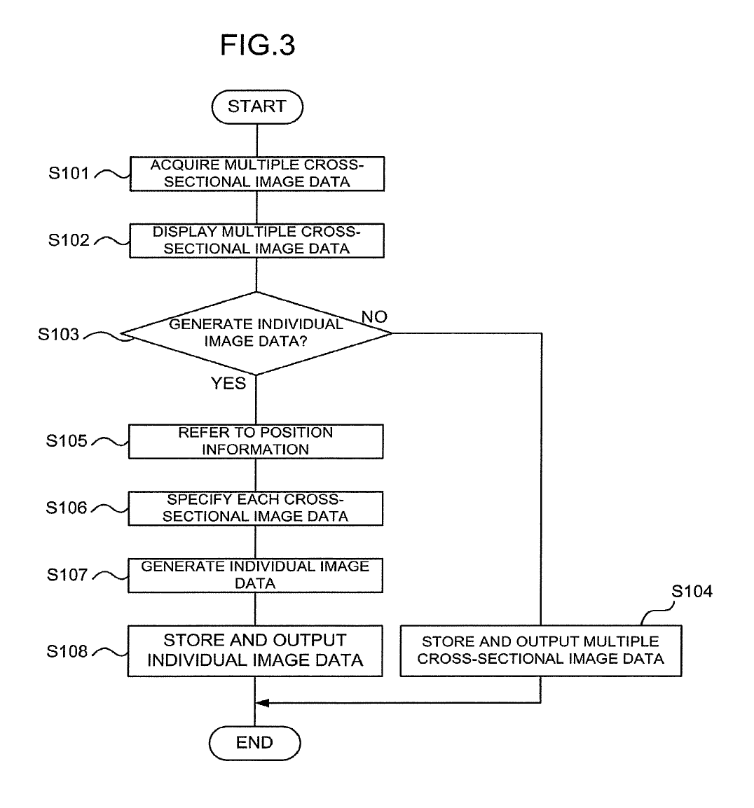

The system control circuit 16 controls each part of the ultrasound diagnosis apparatus based on an operation input signal received through the input circuit 2 and a medical image processing program stored in advance. For example, the medical image processing program stored in the system control circuit 16 implements, when executed, a medical image processing method corresponding to the operation illustrated in the flowchart of FIG. 3.

FIG. 3 is a flowchart illustrating the operation of the ultrasound diagnosis apparatus according to the first embodiment.

Step S101: The operator operates the input circuit 2 and the ultrasound probe 100 to acquire multiple cross-sectional image data. Accordingly, the multiple cross-sectional image generating circuit 112 generates multiple cross-sectional image data indicating a plurality of cross sections of the subject. At this time, the multiple cross-sectional image generating circuit 112 adds position information to the multiple cross-sectional image data while associating each cross section indicated by the position information with a corresponding cross-sectional image data.

Step S102: The multiple cross-sectional image generating circuit 112 outputs the multiple cross-sectional image data generated to the display control circuit 12. The display control circuit 12 displays a plurality of cross-sectional images acquired simultaneously in parallel in one display window on the display 3 as in the case of general multi-plane images.

Step S103: Having received a predetermined setting instruction, the system control circuit 16 determines to generate individual image data (Yes in step S103). In this case, the process proceeds to step S105. When the system control circuit 16 has not received the setting instruction, the circuit 16 determines not to generate individual image data (No in Step S103). In this case, the process proceeds to step S104.

Step S104: As in the case of normal multi-plane scanning, the memory circuit 13 stores the multiple cross-sectional image data. The communication interface 15 outputs the multiple cross-sectional image data to the server 200 in the same manner as in normal multi-plane scanning.

Step S105: The system control circuit 16 controls the multiple cross-sectional image generating circuit 112 to output selected multiple cross-sectional image data to the individual image generating circuit 14. The individual image generating circuit 14 refers to the position information in the multiple cross-sectional image data received from the multiple cross-sectional image generating circuit 112.

Step S106: The individual image generating circuit 14 specifies the number of cross sections contained in the multiple cross-sectional image data, the position of each cross section, and image data associated with each cross section based on the position information.

Step S107: The individual image generating circuit 14 generates individual image data for each cross section and adds thereto position information associated with the image data specified.

Step S108: The individual image generating circuit 14 outputs the individual image data generated to the memory circuit 13 and the communication interface 15. The memory circuit 13 stores the individual image data. The communication interface 15 outputs the individual image data to the server 200.

According to the first embodiment, the ultrasound diagnosis apparatus generates individual image data that enables one cross-sectional image to be displayed in one display window in conjunction with the acquisition of multiple cross-sectional image data in a multi-plane scan examination. This facilitates comparative analysis in a general-purpose workstation.

Further, with the ultrasound diagnosis apparatus of the embodiment, the individual image data can be generated by simple operation without need of once displaying the image and checking its quality. This reduces the time required for the examination. For example, in stress echocardiography, the heart rate rises after stress is applied, and it usually transits in a relatively short time of about 1 to 2 minutes until returning to a normal heart rate. The examination can be conducted easily even in such a short time. In the same way, it is possible to reduce the time taken by other multi-plane scan examinations. In addition, it becomes easy to compare and analyze image data of a plurality of cross sections also in a general workstation which does not have dedicated application software corresponding to image data by multi-plane.

Further, according to the embodiment, the ultrasound diagnosis apparatus generates individual image data for each cross section. Thus, it is easy to select unnecessary individual image data and delete it at the time of subsequent comparative analysis.

### Second Embodiment

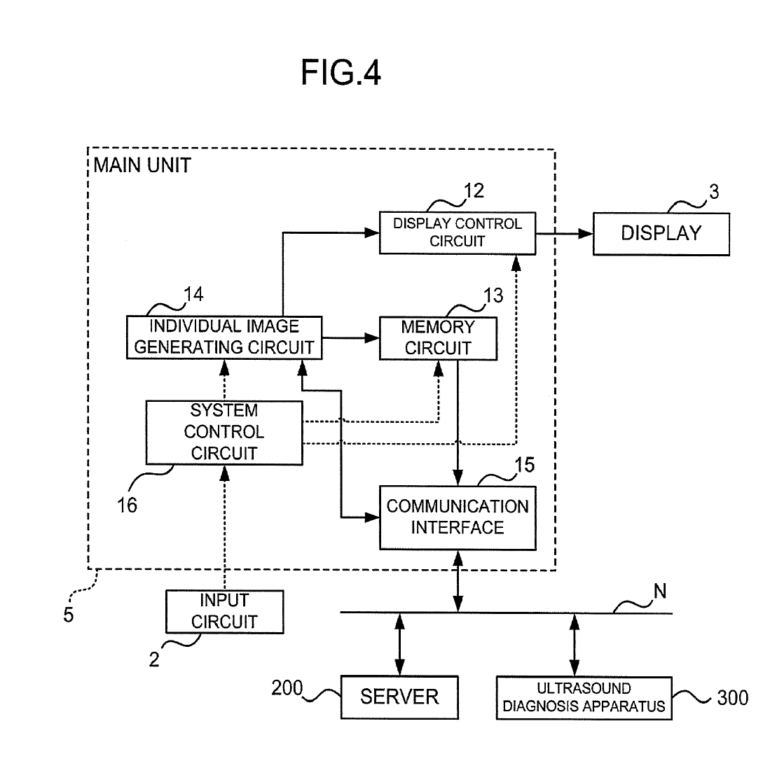

The second embodiment involves a medical image processing apparatus. FIG. 4 is a block diagram illustrating a configuration of a medical image processing apparatus according to the second embodiment. In the following, differences from the first embodiment are mainly described. The medical image processing apparatus of the second embodiment facilitates the comparative analysis of multiple cross-sectional image data, which is ultrasound image data acquired in advance to display a plurality of cross sections simultaneously and concurrently. The medical image processing apparatus of the second embodiment includes a main unit 5, the input circuit 2, and the display 3, and is communicably connected to the server 200 and an ultrasound diagnosis apparatus 300.

The server 200 stores plural cross-sectional image data acquired in advance. In the multiple cross-sectional image data, image data of each cross section and position information of the cross section are grouped into one set.

The communication interface 15 reads the multiple cross-sectional image data from the server 200 or the ultrasound diagnosis apparatus 300 via the network N. The communication interface 15 outputs the multiple cross-sectional image data to the individual image generating circuit 14.

As in the first embodiment, the individual image generating circuit 14 refers to the position information of the multiple cross-sectional image data. Through this reference, the individual image generating circuit 14 specifies the number of cross sections contained in the multiple cross-sectional image data, the position of each cross section, and image data associated with each cross section.

The individual image generating circuit 14 generates individual image data while adding thereto the position information associated with the image data specified. The individual image generating circuit 14 performs this generation process for each cross section contained in the multiple cross-sectional image data.

The individual image generating circuit 14 may output each piece of image data to the display control circuit 12 and generate individual cross-sectional image data while displaying the multiple cross-sectional image data and individual cross-sectional image data already generated on the display 3. With this, the operator can operate the medical image processing apparatus while viewing the images.

The individual image generating circuit 14 outputs the individual image data thus generated to the memory circuit 13 and the communication interface 15. The memory circuit 13 stores the individual image data. The communication interface 15 outputs the individual image data to the server 200.

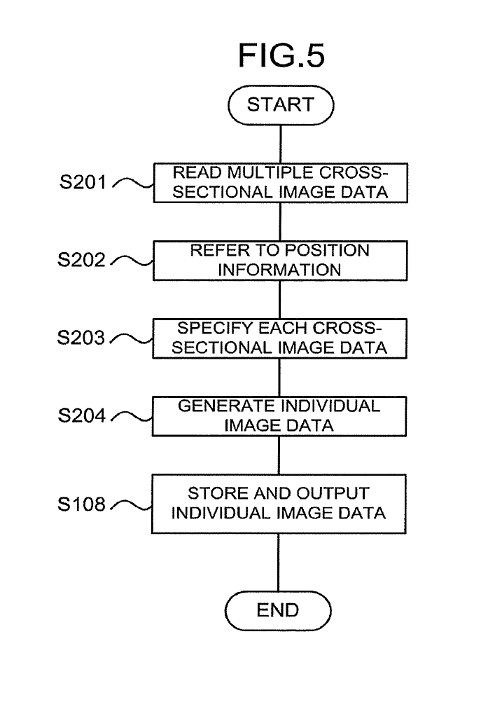

FIG. 5 is a flowchart illustrating the operation of the medical image processing apparatus according to the second embodiment.

Step S201: The communication interface 15 reads multiple cross-sectional image data from the server 200 or the ultrasound diagnosis apparatus 300 via the network N. The communication interface 15 outputs the multiple cross-sectional image data to the individual image generating circuit 14.

Step S202: The individual image generating circuit 14 refers to the position information of the multiple cross-sectional image data received from the communication interface 15.

Step S203: The individual image generating circuit 14 specifies the number of cross sections contained in the multiple cross-sectional image data, the position of each cross section, and image data associated with each cross section based on the position information.

Step S204: The individual image generating circuit 14 generates individual image data for each cross section while adding thereto the position information associated with the image data specified.

Step S205: The individual image generating circuit 14 outputs the individual image data to the memory circuit 13 and the communication interface 15. The memory circuit 13 stores the individual image data. The communication interface 15 outputs the individual image data to the server 200.

According to the second embodiment, the medical image processing apparatus can generate individual image data for each cross section from multiple cross-sectional image data having one set of ultrasound images of a plurality of cross sections obtained by multi-plane examination, which is acquired and stored in advance. Thereby, it is also possible to facilitate the comparative analysis of the multiple cross-sectional image data acquired and stored in advance.

While the comparative analysis in the stress echo examination has been described herein, the ultrasonic wave diagnostic apparatus, the medical image processing apparatus, and the medical image processing method of the embodiments may be applied to other examinations. Examples of the examinations include various multi-plane examinations for acquiring a plurality of cross-sectional images, various ultrasound examinations in which data needs to be acquired in a short time due to restriction on scan time for capturing images.

With the ultrasound diagnosis apparatus, the medical image processing apparatus, and the medical image processing method according to at least one embodiment described above, it is possible to facilitate the comparative analysis of multiple cross-sectional image data acquired by a multi-plane probe.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus, comprising processing circuitry configured to:
acquire multiple cross-sectional image data that are ultrasound image data for simultaneously displaying a plurality of cross sections of a subject; and
generate individual image data for each of the cross sections based on position information of the cross section as well as displaying the multiple cross-sectional image data.

2. The ultrasound diagnosis apparatus of claim 1, further comprising a communication interface configured to output the individual image data to an external medical image processing apparatus.

3. The ultrasound diagnosis apparatus of claim 1, wherein the processing circuitry is further configured to
acquire the multiple cross-sectional image data while adding thereto cross-section direction information indicating direction of each of the cross sections as the position information, and
generate the individual image data based on the cross-section direction information.

4. The ultrasound diagnosis apparatus of claim 1, wherein the processing circuitry is further configured to
acquire the multiple cross-sectional image data while adding thereto coordinate information indicating coordinates of each of the cross sections as the position information, and
generate the individual image data based on the coordinate information.

5. The ultrasound diagnosis apparatus of claim 1, wherein the processing circuitry is further configured to generate the individual image data while adding thereto individual position information indicating position of a cross section corresponding to the individual image data based on the position information.

6. The ultrasound diagnosis apparatus of claim 1, further comprising an operation unit,
wherein the processing circuitry is further configured to generate the individual image data in response to a predetermined setting instruction provided through the operation unit.

7. The ultrasound diagnosis apparatus of claim 1, further comprising:
a storage configured to store the individual image data; and
a communication interface configured to output the individual image data to an external device.

8. The ultrasound diagnosis apparatus of claim 1, wherein the individual image data is comparative analysis image data for stress echo examination.

9. A medical image processing apparatus, comprising processing circuitry configured to:
read multiple cross-sectional image data that are ultrasound image data acquired in advance for displaying a plurality of cross sections simultaneously; and
generate individual image data for each of the cross sections based on position information of the cross section.

10. A medical image processing method, comprising:
acquiring multiple cross-sectional image data that are ultrasound image data for simultaneously displaying a plurality of cross sections of a subject; and
generating individual image data for each of the cross sections based on position information of the cross section as well as displaying the multiple cross-sectional image data.

11. A medical image processing method, comprising:
reading multiple cross-sectional image data that are ultrasound image data acquired in advance for displaying a plurality of cross sections simultaneously; and
generating individual image data for each of the cross sections based on position information of the cross section.
